(19) United States
(12) Patent Application Publication
(10) Pub. No.: US 2011/0087436 A1
(43) Pub. Date: Apr. 14, 2011

(54) METHOD AND SYSTEM FOR ANALYSIS OF TIME-SERIES MOLECULAR QUANTITIES

(76) Inventors: Maria Klapa, Piraeus (GR); Bhaskar Dutta, Houston, TX (US)

(21) Appl. No.: 12/094,087

(22) PCT Filed: Nov. 17, 2006
(86) PCT No.: PCT/US2006/044536

§ 371 Date: Feb. 2, 2009

Related U.S. Application Data
(60) Provisional application No. 60/737,585, filed on Nov. 17, 2005.

(51) Int. Cl. G06F 19/00; G01N 33/48

(52) U.S. Cl. 702/19

(57) ABSTRACT

A method and system for analyzing a plurality of groups of time-series gene expressions including determining a time-dependent score that comprises a difference between the gene expression in the first group at a given time point and the gene expression in the second group at the given time point, determining an expected difference parameter that comprises a difference between a mean expression of the gene in a third group of samples and a mean expression of the gene in a fourth group of samples, created by randomly dividing the samples into two equal groups, comparing a difference between the time-dependent score and the expected difference parameter to a threshold at the given time point, determining the significant genes at each time point on the basis of the comparison.

This application claims priority from U.S. Provisional Patent Application Ser. No. 60/737,585 entitled “Hypothesis-Testing Based methodology for the Analysis of Time-Series Transcriptomic Data”, filed Nov. 17, 2005. This application is incorporated by reference herein in its entirety.

### BACKGROUND OF THE INVENTION

1. Field of Invention

The present invention relates to statistical analysis of gene related data and, in particular, to a systematic analysis of time-series data that allows for the identification of differentially expressed genes or gene products, or any other molecular quantities measured in a high-throughput manner between various sets of physiological conditions.

2. Description of Related Art

Different biological systems may be characterized by differences in the copy number of genes or in levels of transcription of particular genes. By measuring such biological phenomena, insight into and possible treatment of, for example, human diseases, may be found.

High-throughput transcriptional profiling analysis using deoxyribonucleic acid (“DNA”) microarrays (Brown and Botstsein, “Exploring the new world of the genome with DNA microarrays,” Nature Genetics 21:33-37, 1999; and Schena et al., “Quantitative monitoring of gene expression patterns with a complementary DNA microarray,” Science 270: 467-470, 1995) is an innovative way to approach questions in the area of life sciences. The high-throughput approach, in general, enables the identification of biological fingerprints that are differentially expressed between two biological examining pool states. This identification is made possible through the classical hypothesis testing methods, t-test (Baldi et al., “A Bayesian framework for the analysis of microarray expression data: regularized t—test and statistical inferences of gene changes,” Bioinformatics 17:509-519, 2001; Wang et al., “Sample size for identifying differentially expressed genes in microarray experiments,” J Comput Biol 11:714-726, 2004) and ANOVA (Draghici et al., “Noise sampling method: an ANOVA approach allowing robust selection of differentially regulated genes measured by DNA microarrays,” Bioinformatics 19:1348-1359, 2003; and Zhao et al., “Improved significance test for DNA microarray data: temporal effects of shear stress on endothelial genes,” Physiol. Genomics 12: 1-11, 2002). T-test is also the basis for the Significance Analysis of Microarrays (SAM) (Tusher et al., “Significance analysis of microarrays applied to the ionizing radiation response,” Proc. Natl Acad. Sci 98: 5116-5121, 2001), which, however, is a nonparametric test and is tailored for transcriptional profiling data. SAM provides the benefit of adjusting the significance threshold and calculating the “False Discovery Rate (FDR),” which is a measure of the number of genes identified as significant by chance in a user-friendly manner.

While useful in the analysis of transcriptional profiling data, the classical hypothesis testing techniques cannot be used for the analysis of time-series data. While dealing with time-series data, these methods treat each time point in a sequence as a different experimental condition. The “history” or sequence of time points (alternatively referred to herein as “time-series data”) is not taken into consideration. Classical statistical methods, such as Moving Average (“MA”), Auto Regressive (“AR”), Auto Regressive Moving Average (“ARMA”), for the analysis of time-series data that have been successfully applied to other fields, cannot be equally effective for modeling transcriptional profiling data in particular, and any other cellular fingerprinting in general. This is true because the number of time points in biological experiments is usually much smaller than the number of variables (e.g., in the case of transcriptional profiling of the number of genes). Therefore, the resulting models are rudimentary, primarily due to the impossibility of estimating the model parameters.

Various additional methods for the analysis of time-series data are known. For example, in one method, continuous curves are fitted to discrete data (Bar-Joseph et al., “Comparing the continuous representation of time-series expression profiles to identify differentially expressed genes,” Proc Natl Acad Sci USA 100:10146-10151(a), 2003). The curve-profiles of two different experimental conditions are examined in sequence based on a particular correlation criterion with the objective of determining whether they are independent or a noisy realization of each other (Bar-Joseph et al., “Continuous Representations of Time Series Gene Expression,” J Comput Biol 10:341-356(b), 2003). In another method for identification of differentially expressed genes from time series data, SAM analysis is used to identify genes that are differentially expressed at each time point (Liu et al., “Global Transcription Profiling Reveals Comprehensive Insights into Hypoxic Response in Arabidopsis,” Plant Physiol. 137:1115-1129, 2005). This method identifies the number of positively and negatively significant genes changing with time.

In most of the above-described methods, however, if an analysis of time-series data is performed, each time point is treated as an independent experiment and the information about the sequence of the time points is generally lost. Moreover, an effective and accurate comparison of time-series data requires that time points are compared with respect to a common reference. None of the above-described methods achieves a comprehensive study of the variability of the differentially expressed genes with time. In the case of a time-series experiment, in which each group of samples represents measurements collected at various time points under a particular set of experimental conditions, the conventional SAM analysis identifies the significant genes based only on their overall score calculated from all time points, not for each time individual point. Accordingly, different expression profiles, such as the ones illustrated in FIG. 1, correspond to identical SAM results even though they vary differently over time, because time-dependent information is not taken into consideration in the conventional SAM analysis. To extract the time-dependent interaction, a time-dependent score capturing the gene expression over time must be defined.

FIG. 1 is an illustration of a gene expression profile comparing gene expression over time to the overall gene expression derived from conventional SAM analysis. In FIG. 1, although the various genes have different scores at various time points, their overall SAM score, based on conventional SAM analysis, is the same. The conventional significance score calculated using a conventional SAM analysis is performed using an equation that takes into account the mean expression of the genes, i.e., the expression of the genes over the entire period of time, not the expression of the genes at the various time points. Accordingly, a conventional SAM analysis fails to capture the significance variability of the expression of the genes. There exists a need in the art, therefore, for methods and systems that provide analysis of time-series data for the identification of differentially expressed genes between various sets of physiological conditions.

### SUMMARY OF THE INVENTION

In light of the above described problems and shortcomings, various exemplary embodiments of the systems and methods of the present invention provide a method for analyzing a plurality of groups of time-series genes, the method including determining, for at least one gene within a first group of samples and a second group of samples, a gene expression at various time points in the first group and in the second group, determining, for the at least one gene, a time-dependent score that comprises a difference between the gene expression in the first group at a given time point and the gene expression in the second group at the given time point, determining an expected difference parameter that comprises a difference between a mean expression of the gene in a third group of samples and a mean expression of the gene in a fourth group of samples, comparing a difference between the time-dependent score and the expected difference parameter to a threshold at the given time point, and determining significant genes on the basis of the comparison.

Also, various exemplary embodiments of the present invention provide a system for analyzing a plurality of groups of time-series genes, the system including means for determining, for at least one gene within a first group of samples and a second group of samples, a gene expression at various time points in the first group and in the second group, means for determining, for the at least one gene, a time-dependent score that comprises a difference between the gene expression in the first group at a given time point and the gene expression in the second group at the given time point, means for determining an expected difference parameter that comprises a difference between a mean expression of the gene in a third group of samples and a mean expression of the gene in a fourth group of samples, means for comparing a difference between the time-dependent score and the expected difference parameter to a threshold at the given time point, and means for determining significant genes on the basis of the comparison.

Finally, various exemplary embodiments of the present invention provide a computer program embodied on a recordable medium, the program including instructions to analyzing a plurality of groups of time-series genes by determining, for at least one gene within a first group of samples and a second group of samples, a gene expression at various time points in the first group and in the second group, determining, for the at least one gene, a time-dependent score that comprises a difference between the gene expression in the first group at a given time point and the gene expression in the second group at the given time point, determining an expected difference parameter that comprises a difference between a mean expression of the gene in a third group of samples and a mean expression of the gene in a fourth group of samples, comparing a difference between the time-dependent score and the expected difference parameter to a threshold at the given time point, and determining significant genes on the basis of the comparison.

### BRIEF DESCRIPTION OF THE DRAWINGS

Various exemplary embodiments of the systems and methods will be described in detail, with reference to the following figures, wherein:

FIG. 1 is an illustration of a gene expression profile comparing gene expression over time to the overall gene expression derived from conventional SAM analysis;

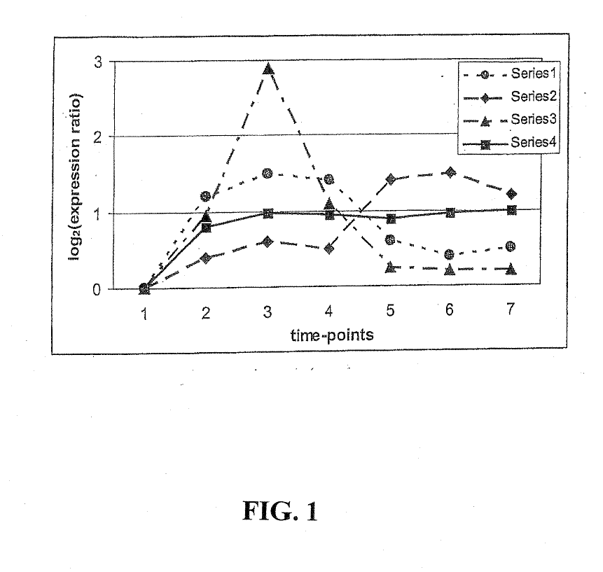

FIG. 2 is a flow chart illustrating an exemplary method for analyzing a plurality of groups of time-series genes;

FIG. 3 is an illustration of matrices used to study the significance variability of the exemplary expression of a gene;

FIG. 4 presents an exemplary system diagram of various hardware components and other features, for use in accordance with an embodiment of the present invention; and

FIG. 5 is a block diagram of various exemplary system components, in accordance with an embodiment of the present invention.

These and other features and advantages of this invention are described in, or are apparent from, the following detailed description of various exemplary embodiments of the systems and methods.

### DETAILED DESCRIPTION OF PREFERRED EMBODIMENTS

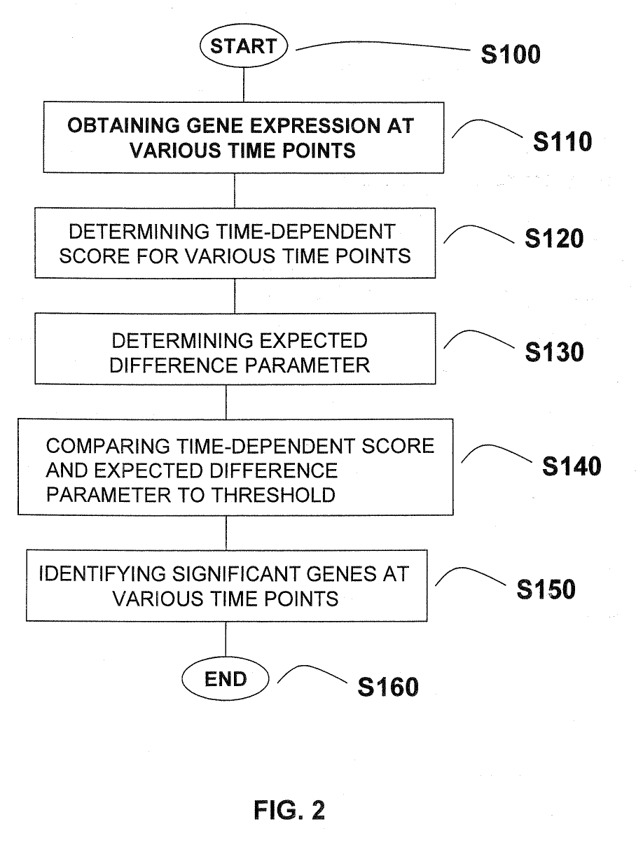

FIG. 2 is a flow chart illustrating an exemplary method for analyzing a plurality of groups of time-series genes. In FIG. 2, the method starts at step S100 and continues to step S110, where a gene expression is obtained at various time points for a gene in a first group of samples and a second group of samples. Gene expression is generally understood to be the process by which a gene DNA sequence is converted into the structures and functions of a cell. Gene expression is generally a multi-step process that begins with transcription of DNA, which genes are comprised of, into messenger ribonucleic acid (“RNA”). It is then followed by post-transcriptional modification and translation into a gene product, such as a protein, followed by folding, post-translational modification and targeting. The amount of protein that a cell expresses depends on the tissue, the developmental stage of the organism and the metabolic or physiologic state of the cell. At step S110, the expression of a given gene is measured in at least two groups of samples. According to various exemplary embodiments, an expression for each gene may be calculated in each group of samples at various points in time, so that a gene expression is recorded at various points in time in, e.g., a first group and a second group of samples. Next, control continues to step S120, where a time-dependent score is determined at the various time points in both groups of samples.

At step S120, a time-dependent score dₜ(i), for gene “i” at time point “t”, is determined on the basis of obtained gene expressions at each of the time points for gene “i” in both the first group and the second group of samples. In the case of a time-series experiment, in which each group of samples represents those collected at the various time points under a particular set of experimental conditions, a SAM analysis may allow the identification of the significant genes based on their overall time-dependent score calculated from all time points, but not the expression of a gene at various points in time. To extract the time-dependent gene expression, a time dependent score dₜ(i) is defined as being the observed score, of gene “i” at time point “t”. It should be noted that each time point under a set of conditions is represented by the geometric mean expression of its replicates.

$${d}_{t} ( i ) = \frac{( {X}_{1}^{t} ( i ) - {X}_{2}^{t} ( i ) )}{S ( i ) + {S}_{0}} \qquad ( 1 )$$

where

X₁^{t} (i) is the expression of gene i at the time point t of the first group of genes;

X₂^{t} (i) is the expression of gene i at the time point t of the second group of genes;

S(i) is the standard deviation of i^{th} gene expression; and

S₀ is a fudge factor, used to eliminate numerical biases at low values of S(i).

Although, in general, the statistic relative difference d(i) should be independent of the gene expression level, at low expression levels, d(i) can be high because of small values of S(i). Thus, in order to eliminate such bias, a small positive constant (S₀) may be added to the denominator of equation (1). Once the time-dependent score is determined during step S120, control continues to step S130, where an overall expected difference parameter is calculated.

During step S130, the overall expected difference parameter dₑ(i) is determined. According to various exemplary embodiments, dₑ(i) is calculated based on the following expression:

$${d}_{e} ( i ) = \frac{( {\overline{X}}_{3} - {\overline{X}}_{4} )}{S ( i ) + {S}_{0}} \qquad ( 2 )$$

where

X̅₃ is the mean expression of gene i in group 3;

X̅₄ is the mean expression of gene i in group 4;

S(i) is the standard deviation of i^{th} gene expression; and

S₀ is a fudge factor, used to eliminate numerical biases at low values of S(i).

In this case, groups 3 and 4 are two groups that are derived from the original first and second group of samples as follows: all the samples in the first group and the second group are assembled as one overall group, and the overall group is then divided randomly into two groups of equal size, which is also the size of the first and second groups, to obtain groups 3 and 4. There are many possible permutations to obtain groups 3 and 4; accordingly, an expected difference dₑ(i) is calculated as indicated in equation (2) for each one of the permutations, and the overall expected difference parameter is determined as the median value of all the calculated expected differences for all the possible permutations. That median value is the overall expected difference parameter. Next, control continues to step S140.

At step S140, a comparison is made between the absolute difference between the time-dependent score and the overall expected difference parameter, and a threshold value. First, the absolute difference is calculated between the time-dependent score d(i) and the overall expected difference parameter dₑ(i). Then, this absolute difference is compared to a threshold value Delta. According to various exemplary embodiments, a given gene is deemed significant when the absolute difference between d(i) and dₑ(i) is higher than the threshold Delta for the given gene. In other words, if the above discussed absolute difference is greater than the threshold Delta, then the gene is significant at that given time point. It should be noted that conventional SAM analysis does not differentiate between two genes that have been identified as significant overall, i.e., over a period of time, but where one gene may have been significant only at one time point, whereas the other gene may have been significant consistently at all the time points. Next, control continues to step S150.

At step S150, significant genes are identified at each time point, on the basis of the comparison made during step S140. The identified significant genes may be stored in a compact form in a matrix which has dimensions corresponding to the number of genes and the number of time points. The significant genes may also be analyzed to determine, for example, the variability of the different genes, a correlation of the different time points of the experiment, or to compare different gene ontology (GO) terms that are significantly different between the two groups. Next, control continues to step S160, where the method ends.

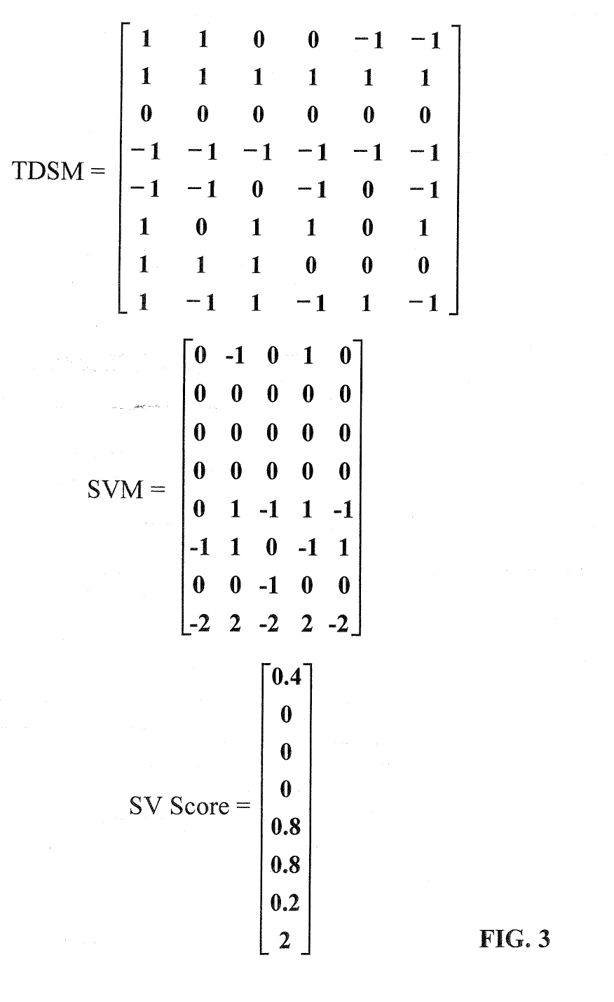

FIG. 3 is an illustration of matrices used to study the significance variability of the exemplary expression of a gene. In FIG. 3, if g and k are the number of genes and the number of time points, respectively, a (g×k) exemplary matrix can be constructed, alternatively referred to herein as a Time-Dependent Significance Matrix (TDSM). In this TDSM matrix, the [i,j]-th element is 1, −1, or 0 depending on whether gene “i” has been identified as positively significant (i.e., an absolute difference between the time-dependent score and the expected overall difference parameter is greater than a threshold Delta), negatively significant or non-significant, respectively at time j. Expression values of genes that are missing at some of the time points may be imputed using different existing data imputation algorithms. It will be apparent to one of ordinary skill in the art, that by using exemplary matrix TDSM, the significance variability of a particular gene expression between time points can be studied. The matrix TDSM may be calculated via a SAM-based methodology, or by developing some other suitable algorithm for finding differentially expressed genes at each time point. According to various exemplary embodiments, 1 and −1 in matrix TDSM are characterizations specified in FIG. 3 and the following description.

Different clustering algorithms may be applied based on matrix TDSM to cluster genes that show similar significance profiles over time. The TDSM matrix can thus be used for clustering in time, alternatively referred to herein as “time space” clustering. Genes that are clustered together show similar differential expressions over time. Sometimes, it may be desirable to study the specific behavior of genes. For example, genes may show either an acute response or a long-term response when subjected to stress. An object of interest may also be genes that are up-regulated at some time points, but down-regulated at other time points. Genes that show cyclic behavior in terms of their differential expression, i.e., become differentially expressed after a certain time interval may also be important for a specific purpose. Knowledge of genes that are differentially expressed at each time point separately allows more precise analysis. As an example, it may be desirable to find genes that are over-expressed at time points t₁, t₂, t₃, under-expressed at time points t₄, t₅ and over-expressed at time points t₆ and t₇. This problem may be mathematically translated as

G=GPₜ₁∩GPₜ₂∩GPₜ₃∩GNₜ₄∩GNₜ₅∩GPₜ₆∩GPₜ₇ (3)

where G is the number of genes found from the analysis; and
GPₜ₁ and GNₜ₁ are the genes that are positively and negatively significant at time point tᵢ and tⱼ respectively. Also, template matching can be used to find genes that show differential expression profiles that are similar to the one of interest, such as the one expressed in equation (3).

Also in FIG. 3, a Significance Variability Matrix (SVM), which is a measure of how the significance level of genes are changing may be constructed as a g×(k−1) matrix. SVM is calculated from the TDSM matrix using the following formula:

SVM[i,(j−1)]=|TDSM[i,j]−TDSM[i,(j−1)]|,

where the 0, 1 and 2 values are selected to represent the number of significance jumps of a particular gene from j^{th} to the (j+1)^{th} time point.

A Significance Variability score vector SV, which is a measure of how variable the significance levels of the genes are over time, may thus be estimated for a set of genes, for each of which the significance level at each time point is reflected in the TDSM matrix. The variability of the significance level for each gene over all of the time points may be computed by adding the absolute values of the elements of a row of the SVM matrix. The SV score, as illustrated in FIG. 3, is estimated as follows:

$$S V [ i ] = \frac{{\sum }_{j = 1}^{{N}_{T} - 1} S V M [ i , j ]}{{N}_{T} - 1} \qquad ( 4 )$$

where

N_{T} is the number of timepoints of the experiment

SV[i] is the i^{th} element of the vector SV.

The SV score enables, for example, the ranking of the genes in order of significance, and thus the derivation of conclusions as to the nature of the genes. Genes with the highest SV scores show the most variability in their differential expressions. In the matrix illustrated in FIG. 3, SV[i]=0, if TDSM [i,j]=1, −1, or 0 at all the time points, and the genes show zero variability in their differential expressions.

A Significance Correlation Matrix (SCM) with respect to positively, negatively or non-significant genes may also be defined as the N_{T}×N_{T} symmetric matrix, whose elements are estimated as follows:

$${SCM}_{k} [ i , j ] = \{ \begin{matrix}\frac{{G}_{k}^{i} ⋂ {G}_{k}^{j}}{\sqrt{{G}_{k}^{i} \cdot {G}_{k}^{j}}} & for i \neq j \\ \frac{\overline{{G}_{k}^{i}}}{{G}_{k}^{i}} & for i = j\end{matrix} \qquad ( 5 )$$

where k depicts the significance level with respect to which the time point comparison is performed (for example, k=P, N, O or P∩N, if the comparison is made with respect to the positively, negatively, non-significant, or the union of positively and negatively significant genes); Gₖ^{l} depicts the number of genes in the k-th significance level at the l-th time point, l=1, 2, . . . , N_{T}, G̅ₖ̅^̅{̅l̅}̅ depicts the number of genes in the k-th significance level only at the l-th time point (i.e G̅ₖ̅^̅{̅l̅}̅∩Gₖ^{q}=0∀q≠l, q=1, 2, . . . , N_{T}).

According to various exemplary embodiments, the elements of a SCM may have values between 0 and 1. Two time points might be considered strongly correlated if the corresponding SCM element is larger than a certain value-threshold, usually larger than 0.5. In addition, a large diagonal element implies that at this time point the response of the system to the particular perturbation is largely different than at the rest.

According to various exemplary embodiments, If a particular GO term is of interest, then the matrices described in the above sections should be constructed to contain only the gene set associated with this GO term; the same analytical methodologies described above could be used to extract biologically relevant conclusions focused only on this GO term. However, to compare GO terms with respect to their differential change in expression with time, a hyper-geometric distribution may be used to compute the GO term enrichment. Assuming that the total number of genes used for an analysis is N, and among them n genes are significant at a particular time point t, if out of y genes that are related to a particular GO term (based on repository of genes used for analysis), x are found significant at the same time point t, then the probability of the event is given by

$$\frac{{}_{}^{y}{C}{}_{x}^{} {}_{}^{( N - y )}{C}{}_{( n - x )}^{}}{{}_{}^{N}{C}{}_{n}^{}} \qquad ( 6 )$$

The null hypothesis (Hₒ) can be created that genes belonging to the GO term i is not significantly enriched, the p value can be computed for that GO term in the following way

$$α = \frac{{\sum }_{i = x}^{y} {}_{}^{y}{C}{}_{i}^{} {}_{}^{( N - y )}{C}{}_{( n - i )}^{}}{{}_{}^{N}{C}{}_{n}^{}} \qquad ( 7 )$$

GO terms that are significantly enriched will pass test criterion (say p<0.05) defined by the user. Specifically, matrices corresponding to each (or to the union of more than one) of the significance levels could be formed; each of the matrices will have as many columns as the number of the sampled time points and as many rows as the number of GO terms that are to be investigated (in a high-throughput unsupervised way, the latter could be all the GO terms that are associated with the gene list under investigation). The [i,j]-th element of a particular significance level's matrix will be equal to the p value of the i-th GO term corresponding to j-th time timepoint. Studying the information in these matrices, it would be possible to answer a variety of questions regarding the response of the various GO terms to the applied perturbation based on their significance level profile over time.

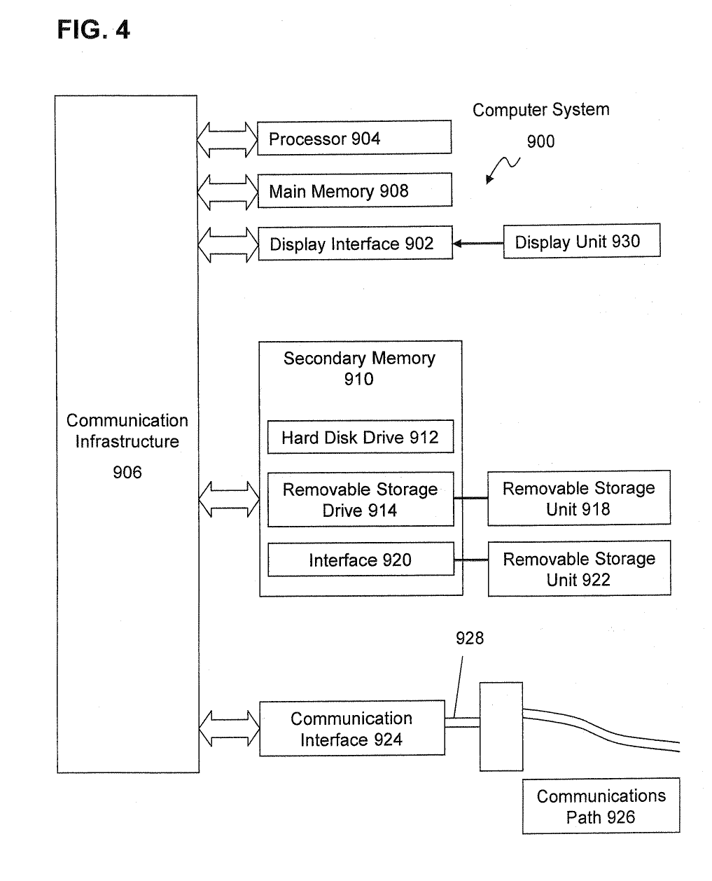

FIG. 4 presents an exemplary system diagram of various hardware components and other features, for use in accordance with an embodiment of the present invention. The present invention may be implemented using hardware, software, or a combination thereof and may be implemented in one or more computer systems or other processing systems. In one embodiment, the invention is directed toward one or more computer systems capable of carrying out the functionality described herein. An example of such a computer system 900 is shown in FIG. 4.

Computer system 900 includes one or more processors, such as processor 904. The processor 904 is connected to a communication infrastructure 906 (e.g., a communications bus, cross-over bar, or network). Various software embodiments are described in terms of this exemplary computer system. After reading this description, it will become apparent to a person skilled in the relevant art(s) how to implement the invention using other computer systems and/or architectures. Computer system 900 can include a display interface 902 that forwards graphics, text, and other data from the communication infrastructure 906 (or from a frame buffer not shown) for display on a display unit 930. Computer system 900 also includes a main memory 908, preferably random access memory (RAM), and may also include a secondary memory 910. The secondary memory 910 may include, for example, a hard disk drive 912 and/or a removable storage drive 914, representing a floppy disk drive, a magnetic tape drive, an optical disk drive, etc. The removable storage drive 914 reads from and/or writes to a removable storage unit 918 in a well-known manner. Removable storage unit 918, represents a floppy disk, magnetic tape, optical disk, etc., which is read by and written to removable storage drive 914. As will be appreciated, the removable storage unit 918 includes a computer usable storage medium having stored therein computer software and/or data.

In alternative embodiments, secondary memory 910 may include other similar devices for allowing computer programs or other instructions to be loaded into computer system 900. Such devices may include, for example, a removable storage unit 922 and an interface 920. Examples of such may include a program cartridge and cartridge interface (such as that found in video game devices), a removable memory chip (such as an erasable programmable read only memory (EPROM), or programmable read only memory (PROM)) and associated socket, and other removable storage units 922 and interfaces 920, which allow software and data to be transferred from the removable storage unit 922 to computer system 900.

Computer system 900 may also include a communications interface 924. Communications interface 924 allows software and data to be transferred between computer system 900 and external devices. Examples of communications interface 924 may include a modem, a network interface (such as an Ethernet card), a communications port, a Personal Computer Memory Card. International Association (PCMCIA) slot and card, etc. Software and data transferred via communications interface 924 are in the form of signals 928, which may be electronic, electromagnetic, optical or other signals capable of being received by communications interface 924. These signals 928 are provided to communications interface 924 via a communications path (e.g., channel) 926. This path 926 carries signals 928 and may be implemented using wire or cable, fiber optics, a telephone line, a cellular link, a radio frequency (RF) link and/or other communications channels. In this document, the terms “computer program medium” and “computer usable medium” are used to refer generally to media such as a removable storage drive 980, a hard disk installed in hard disk drive 970, and signals 928. These computer program products provide software to the computer system 900. The invention is directed to such computer program products.

Computer programs (also referred to as computer control logic) are stored in main memory 908 and/or secondary memory 910. Computer programs may also be received via communications interface 924. Such computer programs, when executed, enable the computer system 900 to perform the features of the present invention, as discussed herein. In particular, the computer programs, when executed, enable the processor 910 to perform the features of the present invention. Accordingly, such computer programs represent controllers of the computer system 900.

In an embodiment where the invention is implemented using software, the software may be stored in a computer program product and loaded into computer system 900 using removable storage drive 914, hard drive 912, or communications interface 920. The control logic (software), when executed by the processor 904, causes the processor 904 to perform the functions of the invention as described herein. In another embodiment, the invention is implemented primarily in hardware using, for example, hardware components, such as application specific integrated circuits (ASICs). Implementation of the hardware state machine so as to perform the functions described herein will be apparent to persons skilled in the relevant art(s). In yet another embodiment, the invention is implemented using a combination of both hardware and software.

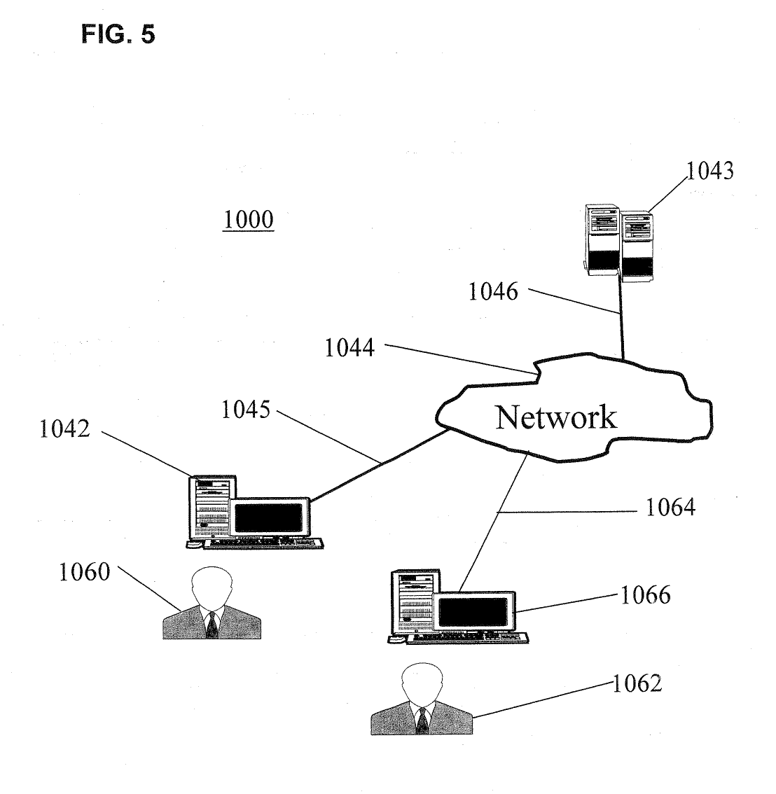

FIG. 5 is a block diagram of various exemplary system components, in accordance with an embodiment of the present invention. FIG. 5 shows a communication system 1000 usable in accordance with the present invention. The communication system 1000 includes one or more accessors 1060, 1062 (also referred to interchangeably herein as one or more “users”) and one or more terminals 1042, 1066. In one embodiment, data for use in accordance with the present invention is, for example, input and/or accessed by accessors 1060, 1064 via terminals 1042, 1066, such as personal computers (PCs), minicomputers, mainframe computers, microcomputers, telephonic devices, or wireless devices, such as personal digital assistants (“PDAs”) or a hand-held wireless devices coupled to a server 1043, such as a PC, minicomputer, mainframe computer, microcomputer, or other device having a processor and a repository for data and/or connection to a repository for data, via, for example, a network 1044, such as the Internet or an intranet, and couplings 1045, 1046, 1064. The couplings 1045, 1046, 1064 include, for example, wired, wireless, or fiberoptic links. In another embodiment, the method and system of the present invention operate in a stand-alone environment, such as on a single terminal.

It will be appreciated that variants of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

What is claimed is:

1. A method for analyzing a plurality of groups of time-series molecular fingerprints including gene expressions, the method comprising:
determining, for at least one gene within a first group of samples and a second group of samples, a gene expression at various time points in the first group and in the second group;
determining, for the at least one gene, a time-dependent score that comprises a difference between the gene expression in the first group at a given time point and the gene expression in the second group at the given time point;
determining an expected difference parameter that comprises a difference between a mean expression of the gene in a third group of samples and a mean expression of the gene in a fourth group of samples;
comparing an absolute difference between the time-dependent score and the expected difference parameter to a threshold at the given time point; and
determining significant genes at the time point on the basis of the comparison.

2. The method of claim 1, wherein the significant genes are genes that are significant relative to other genes.

3. The method of claim 1, wherein the time-dependent score is expressed by:
$${d}_{t} ( i ) = \frac{( {X}_{1}^{t} ( i ) - {X}_{2}^{t} ( i ) )}{S ( i ) + {S}_{0}} ,$$
wherein
X₁^{t} (i) is the expression of gene i at the time point t of the first group;
X₂^{t} (i) is the expression of gene i at the time point t of the second group;
(Xᵢ^{t}(i)-X₂^{t}(i)) represents the difference in the expression of gene i between the two experimental groups at timepoint t;
S(i) is a standard deviation of the expression of gene i; and
S₀ is a fudge factor.

4. The method of claim 1, wherein the third and fourth groups of samples are obtained via random sampling permutations of the samples in the first and second groups, which are first grouped in one larger group and then split into the third and the fourth groups, and the third and fourth groups are of the same size as the first and second groups.

5. The method of claim 4, wherein, for each permutation of the samples, a difference parameter is determined to be a difference between the mean expression of the at least one gene in the third group and in the fourth group.

6. The method of claim 5, wherein the difference parameter for each permutation is determined as
$${d}_{e} ( i ) = \frac{( {\overline{X}}_{3} - {\overline{X}}_{4} )}{S ( i ) + {S}_{0}} ,$$
where
X̅₃ is the mean expression of gene i in the third group;
X̅₄ is the mean expression of gene i in the fourth group;
S(i) is a standard deviation of the expression of gene i; and
S₀ is a fudge factor.

7. The method of claim 6, wherein the expected difference parameter is determined to be a median of the difference parameters for all the permutations.

8. The method of claim 1, wherein the first group is a control group and the second group is a study group.

9. The method of claim 1, wherein the difference between the time-dependent score and the expected difference parameter is an absolute difference.

10. The method of claim 1, wherein the significant genes are correlated to a differential expression of the genes.

11. A system for analyzing a plurality of groups of time-series genes, the system comprising:
means for determining, for at least one gene within a first group of samples and a second group of samples, a gene expression at various time points in the first group and in the second group;
means for determining, for the at least one gene, a time-dependent score that comprises a difference between the gene expression in the first group at a given time point and the gene expression in the second group at the given time point;
means for determining an expected difference parameter that comprises a difference between a mean expression of the gene in a third group of samples and a mean expression of the gene in a fourth group of samples;
means for comparing a difference between the time-dependent score and the expected difference parameter to a threshold at the given time point; and
means for determining significant genes at the time point on the basis of the comparison.

12. The system of claim 11, further comprising:
means for correlating the significant genes to a differential expression of the genes.

13. A computer program embodied on a recordable medium, the program comprising instructions for analyzing a plurality of groups of time-series genes by:
determining, for at least one gene within a first group of samples and a second group of samples, a gene expression at various time points in the first group and in the second group;
determining, for the at least one gene, a time-dependent score that comprises a difference between the gene expression in the first group at a given time point and the gene expression in the second group at the given time point;
determining an expected difference parameter that comprises a difference between a mean expression of the gene in a third group of samples and a mean expression of the gene in a fourth group of samples;
comparing an absolute difference between the time-dependent score and the expected difference parameter to a threshold at the given time point; and
determining significant genes at the time point on the basis of the comparison.

14. The computer program of claim 13, further comprising instructions by:
correlating the significant genes to a differential expression of the genes.
